(19) United States
(12) Patent Application Publication
(10) Pub. No.: US 2019/0357971 A1
(43) Pub. Date: Nov. 28, 2019

(54) DEVICE, SYSTEM AND METHOD FOR SAFE RADIO FREQUENCY TREATMENT

(71) Applicant: Viora Ltd., Netanya (IL)

(72) Inventors: Ofer ADI, Ramat Gan (IL); Eliran ALMOG, Regba (IL)

(73) Assignee: Viora Ltd., Netanya (IL)

(21) Appl. No.: 15/985,740

(22) Filed: May 22, 2018

(51) Int. Cl. A61B 18/14; A61B 18/18

(52) U.S. Cl. CPC A61B 18/1477; A61B 2018/1823; A61B 18/1815

(57) ABSTRACT

A device, a system and a method for applying radio frequency (RF) treatment to a tissue, the including: (a) contacting the tissue with an RF emission portion comprising: a first electrode, having a first contact surface and a second electrode having a second contact surface that is larger than the first contact surface and (b) emitting at least one RF current pulse from the first contact surface, through the treated tissue, to the second contact surface.

### FIELD OF THE INVENTION

The present invention relates to the field of non-surgical treatment. More particularly, the present invention relates to the field of radio frequency (RF) treatment.

### BACKGROUND OF THE INVENTION

The benefits of Radio Frequency (RF) treatment have been studied in relation to numerous medical and cosmetic applications.

In cosmetics, conduction of mild RF current through certain body tissues has been shown to have contributed to tightening of facial skin, removal of cellulitis and improvement of contour reshaping.

In medical applications, RF treatments have been applied, for example to vaginal tissue, to improve vaginal laxity after vaginal delivery.

Application of RF treatment requires vigorous monitoring of physical parameters at the position of contact between the RF treatment appliance (e.g. an electrode) and the patient's tissue, as RF treatment constantly involves a threat of damage to the treated tissue, by inappropriate conduction of current through the treated tissue.

### SUMMARY OF THE INVENTION

There is thus provided, in accordance with some embodiments of the invention, a device for radio frequency (RF) treatment of a tissue. The device may include a hand-held portion and an RF emission portion coupled to the hand-held portion. The RF emission portion may include: a first electrode, having a first contact surface area and a second electrode having a second contact surface area that is larger than the first contact surface area. The first electrode may be configured to emit at least one RF current pulse from the first contact surface, through the tissue to be treated, to the second contact surface. In some embodiments, the treated tissue may be a vaginal tissue.

In some embodiments, the device may include an insulating medium, placed between the first electrode and the second electrode. The second electrode may serve as a reference voltage node to the first electrode, and the contact surface of the second electrode may be larger than the contact surface of the first electrode by at least a factor of 3.

The device may include at least one first sensor, configured to measure the value of at least one electric parameter of the treated tissue. The parameter may be selected from the group consisting of: voltage, current, power, energy, resistance, impedance, and combinations thereof. The device may include a second sensor, configured to measure the temperature of the treated tissue.

According to some embodiments, the hand-held portion may include a hollow portion, having an opening toward the treated tissue. The hand-held portion may be configured to accommodate a liquid and may be associated with an actuator. The actuator may be configured to eject at least part of the liquid out of the hollow portion, through the opening onto the treated tissue.

Embodiments may include two or more interchangeable RF emission portions. A first RF emission portion and a second RF emission portions may differ by at least one physical property, including at least one of: a ratio between the area of contact surfaces of the first and second electrodes, a size of at least one of first and second electrodes and a shape of at least one of first and second electrodes.

Embodiments may include a system for RF treatment of a tissue, the system including a hand-held portion and an RF emission portion coupled to the hand-held portion. The RF emission portion may include: a first electrode, having a first contact surface and a second electrode having a second contact surface that may be larger than the first contact surface. The first electrode may be configured to emit at least one RF current pulse from the first contact surface, through the treated tissue, to the second contact surface.

Embodiments may include at least one RF generator, configured to produce at least one RF electric current pulse, and provide the at least one pulse to the RF emission portion. The RF emission portion may be configured to apply the at least one pulse, or a derivative thereof to the treated tissue via the electrodes.

Embodiments of the system may include a processor, communicatively connected to the RF generator. The processor may be configured to control a status of the RF generator, which may be one of active and non-active. The processor may further be configured to control at least one value of a physical parameter of the at least one RF current pulse produced by the RF generator. The at least one physical parameter may be one of a set of physical parameters, including at least one of: RF frequency, RF pulse duration and RF current amplitude.

Embodiments may include at least one first sensor, configured to measure the value of at least one electric parameter of the treated tissue. The measured parameter may be at least one of: voltage, current, power, energy, resistance and impedance. Embodiments may include at least one second sensor, configured to measure the temperature of the treated tissue.

According to some embodiments, the processor may be communicatively connected to at least one sensor. The processor may be configured to obtain measured data therefrom and may control at least one of (a) the status of the RF generator and (b) the value of the at least one physical parameter according to the obtained measured data.

According to some embodiments, the processor may be configured to: (a) receive from a user, via a user interface, a required set of physical parameters, corresponding to a required RF current pulse; (b) control the RF generator to produce a first RF current pulse, having a set of test physical parameters; (c) obtain at least one measurement of at least one electric parameter of the treated tissue from at least one sensor. If the value of the at least one electric parameter is within a predefined range, then the processor may control the RF generator to produce at least one second RF current pulse, having a second set of physical parameters, according to the required set of physical parameters. If the value of the at least one electric parameter is beyond the predefined range, the processor may control the RF generator to not produce the at least one second RF current pulse.

The processor may be configured to monitor, during the production of the at least one second RF current pulse, a value of at least one electric parameter of the treated tissue, as measured by the at least one sensor. If the value of the at least one electric parameter is beyond the predefined range, the processor may control the RF generator to halt the production of the at least one second RF current pulse.

The processor may be configured to control the RF generator to adjust at least one value of a physical parameter of the at least one second RF current pulse, according to (a) the monitored value of at least one electric parameter of the treated tissue, and (b) the required set of physical parameters.

The hand-held portion may include a hollow portion having an opening toward the treated tissue and configured to accommodate a liquid, and an actuator, associated with the hollow portion, and electrically connected to the processor. The actuator may be configured to, upon receiving a command from the processor, eject at least part of the liquid out of the hollow portion, through the opening onto the treated tissue.

Embodiments may include a method of applying RF treatment to a tissue. The method may include contacting the tissue with an RF emission portion including: a first electrode, having a first contact surface and a second electrode having a second contact surface that is larger than the first contact surface and; emitting at least one RF current pulse from the first contact surface, through the treated tissue, to the second contact surface.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic block diagram, depicting a system for radio frequency (RF) treatment system according to some embodiments of the present invention;

FIG. 2A, FIG. 2B and FIG. 2C are respectively a front view, a top view and lateral view of a device for RF treatment, which may be included in a system for RF treatment, according to some embodiments;

FIG. 3 is a time-based waveform of an example to an RF signal, which may be emitted by a system for RF treatment according to some embodiments;

FIG. 4 is a schematic block diagram, depicting components of an RF generator, which may be included in a system for RF treatment, according to some embodiments;

FIGS. 5A and 5B jointly show a flow diagram, depicting the functionality of an RF treatment system according to some embodiments of the present invention; and

FIG. 6 depicts a flow diagram, depicting a method for employing an RF treatment system according to some embodiments of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

Embodiments of the present invention disclose a method, a device and a system for applying RF treatment, by safely conducting RF current pulses between a first and a second electrode, through a patient's bodily tissue. The system is configured to apply the RF treatment, for instance to a patient's vagina, by inserting a treatment module therein, and conducting said electrical RF current pulses through that tissue. “Treatment” is used herein to refer broadly to applying RF energy to bodily tissue for any purpose.

The system may be configured to continuously and/or repetitively measure and monitor physical properties in the vicinity of, and/or in between the first and second electrodes, associated with the treated tissue and the current pulses conducted, therethrough. The system may be configured to continuously control the emission of said current pulses within a predefined safe range, according to the said monitored physical properties, to avoid afflicting damage to the treated tissue.

The measurements may be performed by at least one sensor, and the system may be configured to stop the emittance of RF current pulses, if at least one measured physical property's value exceeds a predetermined threshold, and/or if a change in the measurement of at least one measured physical property's value exceeds a predetermined threshold.

According to some embodiments, the system may be divided to at least two, sub-units, each hosting different functionality. For example, the system may include at least two of: (a) a user interface sub unit, configured to enable a user to set up specific parameters, such as RF frequency, RF current amplitude and RF current pulse width, (b) a back-end sub unit, configured to produce RF current pulses, monitor physical properties of the current pulses and treated tissue, and control the emission of RF current pulses according to said monitored physical properties; and (c) a treatment sub unit, configured to contact a tissue of a patient and apply RF current pulses thereupon.

This functionality may be distributed differently among the different physical sub units. For example, the user interface sub unit may be incorporated within the back-end sub unit. In another example, the function of monitoring and controlling the emission of RF current pulses may be incorporated within the treatment sub unit.

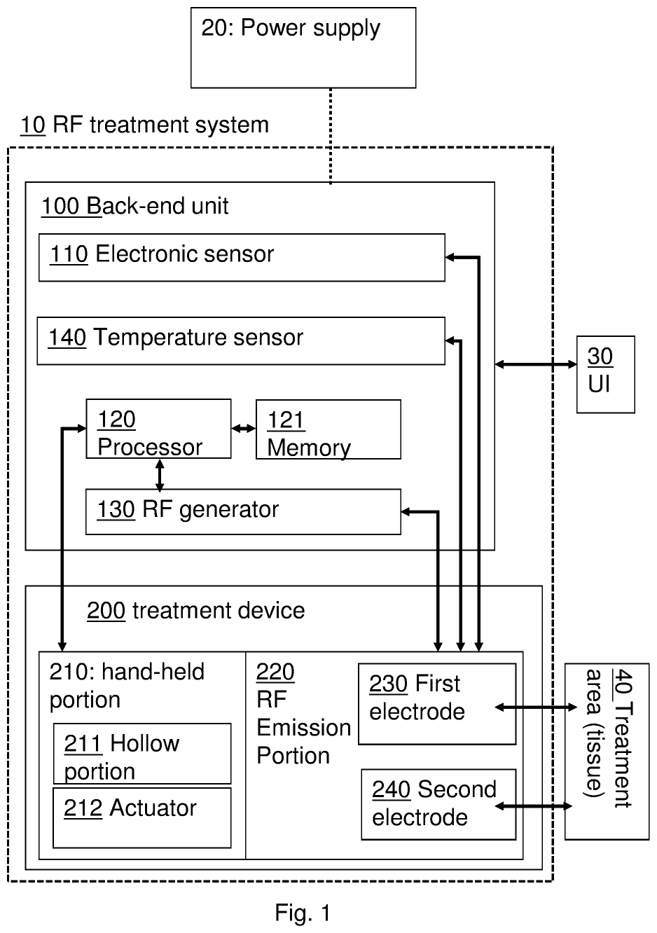

Reference is now made to FIG. 1, showing a block diagram, depicting components of an RF treatment system 10, according to some embodiments. The direction of arrows, as depicted in FIG. 1 describe the direction of data flows between entities. In these embodiments, RF treatment system 10 may include two physical units: a back-end unit 100 and a treatment device 200.

According to some embodiments, treatment device 200 may include at least one of: a hand-held portion 210, and an RF emission portion 220. RF emission portion 220 may include a first electrode 230 having a first contact surface (area in contact with or in close proximity to tissue 40 to be treated), and a second electrode 240 having a second contact surface (area in contact with or in close proximity to tissue 40 to be treated).

In preferred embodiments, the second contact surface, of second electrode 240 may be larger than the first contact surface of first electrode 230. For example, when applying the treatment to a vaginal tissue, second contact surface may be larger than first contact surface by a factor of 10, to ensure a safe and painless route for returning current. In another example, when applying the treatment to less sensitive regions of the body (e.g. on the hands), the second contact surface may be larger than first contact surface by a factor of 3.

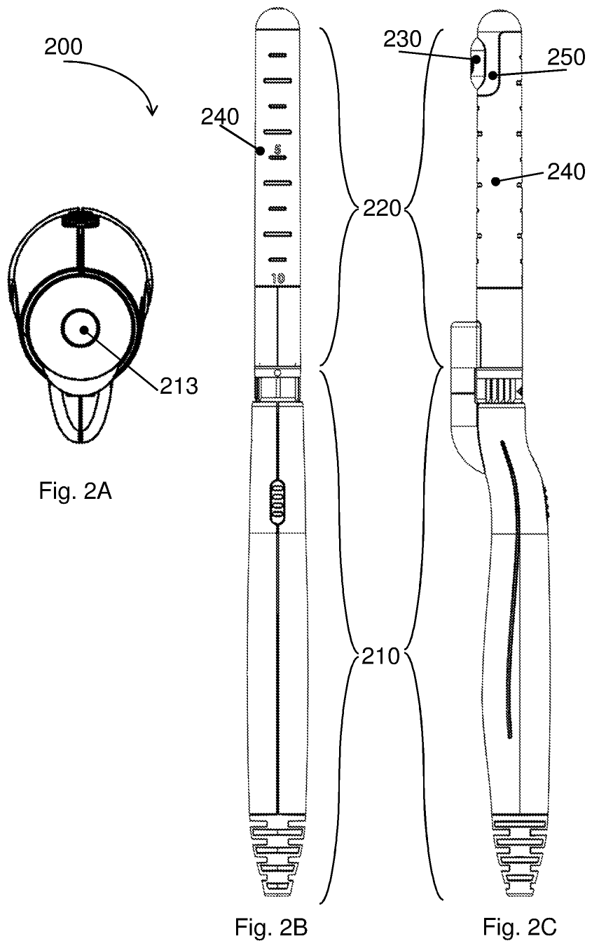

The hand-held portion 210 may be configured to enable comfortable handling of the treatment device, as explained below, in relation to FIGS. 2A, 2B and 2C.

Hand-held portion 210 may include a hollow portion 211 configured to accommodate a liquid and having an opening (e.g. element 213 of FIG. 2A) toward the treated tissue 40. Hand-held portion 210 may be configured to inject the liquid out of the hollow portion (e.g. through a nozzle), onto the treated tissue for the purpose of mitigating excessive heating of treated tissue 40.

In some embodiments, hand-held portion 210 may include an actuator 212, associated with hollow portion 211. Actuator 212 may be configured to eject at least part of the liquid out of the hollow portion, through the opening onto the treated tissue 40. For example, RF treatment system 10 may include a processor, configured to monitor at least one physical parameter (e.g. temperature) of treated tissue 40. Actuator 212 may be configured to receive a command from the processor and eject the liquid according to the command in response to excessive heating (e.g. eject a specific amount of liquid, when the monitored temperature reaches a predefined value).

RF emission portion 220 may be removably coupled to hand-held portion 210 and may be configured to convey electrical RF current pulses to at least two electrodes: the first electrode 230 and the second electrode 240. In some embodiments, a first RF emission portion 220 may be interchangeable with a second RF emission portion, where the first and second RF emission portions may differ by at least one physical property. For example, the first and second RF emission portions may differ by at least one of: a ratio between the area of contact surfaces of the first and second electrodes, a size of at least one of first and second electrodes and a shape of at least one of first and second electrodes.

The first electrode 230 may be configured to contact bodily tissue 40 (e.g. a vaginal tissue) and convey RF current pulses for RF treatment. For example, first electrode 230 may be configured to emit at least one RF current pulse from a first contact surface, through treated tissue 40, to the second contact surface.

According to some embodiments, second electrode 240 may have a contact surface that is larger than the contact surface of the first electrode and may be configured to serve as a reference voltage node to the first electrode. For example, first electrode 230 may emit monopolar RF energy, and second electrode 240 may serve as a ground pad for first electrode 230.

According to some embodiments, second electrode 240 may have a contact surface larger than that of the first electrode 230, e.g. by a factor of 3. Such a factor may provide a dual benefit: (a) The current density at the vicinity of the first electrode may be substantially higher than that of the second electrode, resulting in localization of heat around the contact point of the first electrode with tissue 40, and accurate positioning of the RF treatment. (b) Poor contact of the second electrode with treated tissue 40 may result in increased dissipation of RF energy through the first electrode's contact point with tissue 40, and possible damage to the tissue at that location. Second electrode 240 may have a large contact surface area to ensure reliable contact of the electrode with the tissue 40 to be treated and may eliminate the risk of such damage. This benefit may be particularly relevant when treatment is applied to hidden body parts, such as the vagina, where the location of the electrodes' contact with tissue 40 is not visible.

As mentioned above, RF emission portions 220 may have different physical properties, and may be replaced to accommodate different requirements. For example: RF emission portions 220 may differ in a distance between the contact surfaces of the first and second electrodes. This may enable a user to select a required distance, by replacing a first RF emission portion 220 with a second RF emission portion 220 and attaching the second RF emission portion 220 to the hand-held portion 210.

In another example, different RF emission portions 220 may have different ratios between the sizes of those contact surfaces. During treatment, these geometric differences may result in different emission of current through tissue 40, characterized by different current amplitudes and different current density profiles. These properties of the dissipated current may affect the intensity and localization of the RF treatment, and are hence proprietary to specific, different types of RF treatments. Having the RF emission portion 220 detachable from the hand-held portion may enable a user to select a geometry of the electrodes (e.g. electrode shapes, contact surfaces' sizes, ratios, and distance between contact surfaces) according to the required treatment. This may enable a user to predetermine the distribution of RF current by selecting an RF emission portion 220 that has a predefined ratio between the contact surfaces of the first and second electrodes, and may produce the required current density through tissue 40.

According to some embodiments, an insulating medium (e.g. a plastic portion, element 250 of FIG. 2C) may be placed between the first electrode and the second electrode, enabling the first electrode to emit RF current pulses via the tissue to the second electrode.

According to some embodiments, back-end unit 100 may include at least one of: an RF generator module 130, an electronic sensor 110, a temperature sensor 140, and a processor 120. In alternate embodiments, any one of RF generator module 130, electronic sensor 110, temperature sensor 140, and processor 120 may be embedded within hand-held portion 210 or RF emission portion 220 of treatment device 200.

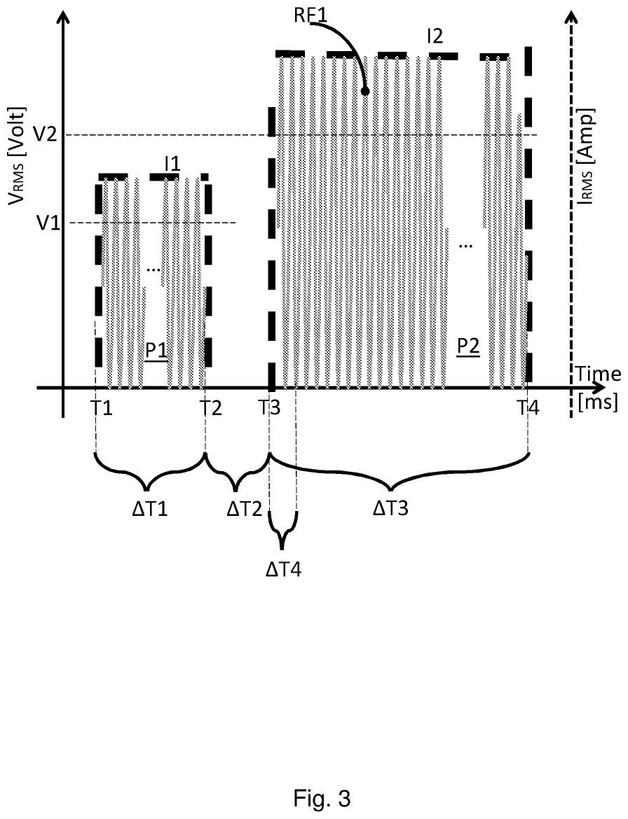

RF generator module 130 may be connected to an electric power supply 20 and may generate at least one RF electric current pulse. RF generator module 130 may provide the at least one pulse (or a derivative thereof) to RF emission portion 220, (as elaborated herein, in relation to FIG. 3). RF emission portion 220 may, in turn, apply the at least one pulse to the treated tissue 40 via electrodes 230 and 240.

RF generator module 130 may generate the at least one RF electric current pulse according to preconfigured parameters, including for example: RF frequency, RF pulse duration and RF current amplitude.

In some embodiments, the preconfigured parameters may be set by a user via a user interface (UI) 30. Alternately, RF generator module 130 may be communicatively connected to processor 120 and may receive commands from processor 120, including required parameters of the RF current pulse. RF generator module 130 may generate the at least one RF electric current pulse according to the received commands, as elaborated herein.

Electronic sensor 110 may be configured to measure the value of at least one electric parameter of the treated tissue, including at least one of a list consisting: voltage, current, dissipated power, dissipated energy, resistance and impedance.

For example, electronic sensor 110 may be a current sensor, including a toroidal coil, configured to determine a value of an electric current, as known to persons skilled in the art of electric engineering.

In some embodiments, electronic sensor 110 may be coupled with the first electrode 230 and may be configured to determine the quality of contact of first electrode 230 with the tissue.

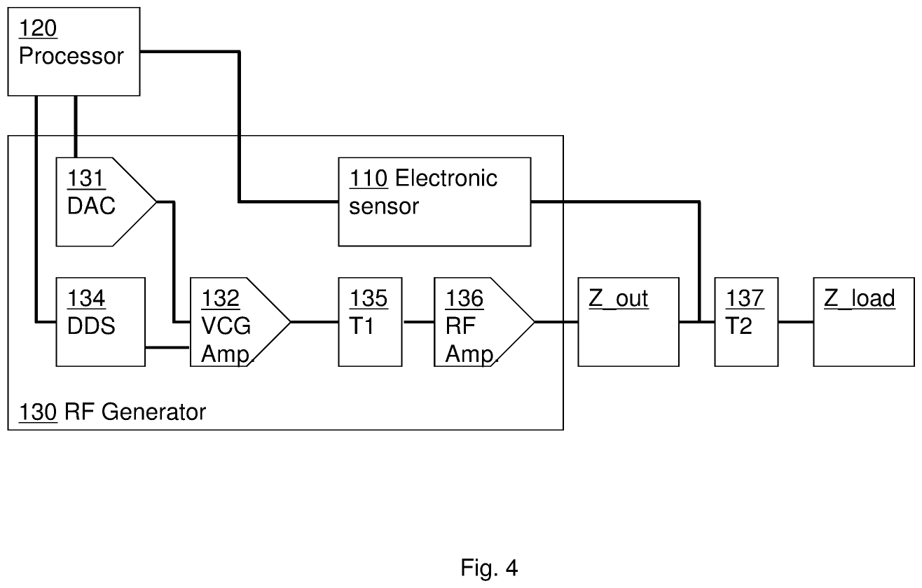

In some embodiments, electronic sensor 110 may be integrated within RF generator module 130, as elaborated in relation to FIG. 4, below.

Electronic sensor 110 may be embedded within treatment device 200, and may be configured to emit at least one signal, indicative of the value of at least one measured electric parameter and propagate the at least one signal to processor 120 for further analysis, as elaborated herein.

Temperature sensor 140 may be embedded within treatment device 200. In some embodiments, temperature sensor 140 may be coupled to first electrode 230 and may be configured to continuously sense the temperature at the location of the first electrode's contact with the tissue. Temperature sensor 140 may be configured to produce signals indicative of the sensed temperature and propagate the signals to processor 120 for further analysis, as elaborated herein.

Processor 120 may be communicatively connected to at least one sensor (e.g. 110, 140) and may obtain measured data therefrom.

For example, processor 120 may be configured to monitor the temperature sensed by temperature sensor 140 and detect conditions of excessive heating of the treated tissue 40 (e.g. when the measure temperature exceeds a predefined threshold). Processor 120 may be electrically connected to actuator 212 of hand-held portion 210 and may control actuator 212 to eject liquid from hollow portion 210 over the treated tissue, to mitigate excessive heating.

Processor 120 may control the status of RF generator 130 (e.g. active and inactive) according to the obtained measured data. For example, if a measured temperature exceeds a predefined threshold, processor 120 may inactivate RF generator 130, to stop providing RF energy to RF emission portion 220 and halt the treatment.

Processor 120 may set a value of at least one physical parameter of the RF pulse generated by RF generator 130 (e.g.: RF frequency, RF pulse duration and RF current amplitude), according to the obtained measured data. For example, processor 120 may receive a nominal value of a required RF current pulse amplitude. Processor 120 may measure a tissue's impedance between the first and second electrode and modify (e.g. increase or decrease) an output voltage of RF generator, to comply with a required RF current pulse amplitude.

In another example, processor 120 may be configured to determine the quality of contact of the first electrode with tissue 40 and control the emission of RF current pulses accordingly. For example, processor may measure at least one electric property (e.g. electric resistance) of the treated tissue. If the electric property (e.g. resistance) is beyond a predefined safety range, processor 120 may control RF generator 130 to halt the generation of RF current pulses.

According to some embodiments, processor 120 may be configured to adapt a user's configuration (e.g. via UI 30) according to data signals received from the electronic sensor 110 and temperature sensor 140. For example, processor 120 may override users' configurations such as power dissipation, and adapt these configurations throughout the treatment, in order to maintain the amplitude of RF current pulses at a predefined, secure range.

Reference is now made to FIG. 2A, FIG. 2B and FIG. 2C, which are, respectively, a front view, a top view and a lateral view of a device for RF treatment 200, which may be included in a system for RF treatment (e.g. element 10 of FIG. 1), according to some embodiments.

As shown in FIGS. 2B and 2C, device 200 may include a hand-held portion 210 and an RF emission portion 220, detachably connected to hand-held portion 210. RF emission portion 220 may be elongated and configured to be inserted into a vagina.

RF emission portion 220 may include a first electrode 230 having a first contact surface, and a second electrode 240, having a second contact surface that is larger than the first contact surface. RF emission portion 220 may include an insulation portion 250, placed between first electrode 230 and second electrode 240. RF emission portion 220 may be configured to convey at least one electric signal from first electrode 230, via a bodily tissue, to second electrode 240.

Hand-held portion 210 may include a hollow portion (not shown), configured to accommodate a liquid, and having an opening 213 toward the treated tissue. Device 200 may be configured to eject at least part of the liquid out of the hollow portion, through the opening onto the treated tissue.

Reference is now made to FIG. 3, which is a time-based waveform of an example for an RF signal, which may be emitted by a system for RF treatment, according to some embodiments.

User interface (UI) module (e.g. element 30 of FIG. 1) may enable a user to configure specific properties of the emitted RF signal according to the required treatment, including for example: an intensity (amplitude) of at least one RF current pulse (e.g. I2), at least one RF pulse width (e.g. ΔT3), at least one RF frequency (e.g. RF1) of an RF pulse, an overall level of energy dissipated by the electrodes, and an overall duration of treatment.

UI module 30 may enable a user to configure an RF signal comprising a plurality of RF current pulses. For example, UI 30 may enable a user to configure a number of RF current pulses in a pulse train (e.g. a number of repetitions of ΔT3), an overall length of a pulse-train (e.g. duration of a pulse-train signal), a duty cycle of a pulse train (e.g. ratio between ΔT2 and ΔT3), etc.

UI module 30 may enable a user to select a specific RF frequency, in order to target a specific tissue layer for treatment, as the dependency of tissue impedance on the dissipated RF energy frequency differs between the layers. In some embodiments, UI 30 may enable a user to select an RF frequency among a plurality of RF frequency configurations within a predefined range. The range of RF frequencies may, for example span between 500 KHz and 3 MHz.

Experimental results have shown that the depth of penetration of effective, therapeutic RF energy is relative to the inverse of a square-root of the RF frequency (e.g. Depth-of-penetration˜1/√(RF-frequency)). Accordingly, a selection of the applied RF frequency may target a different tissue for treatment.

UI module 30 may enable a user to select at least one work mode of RF generator 130 from a plurality of work modes. The work modes may, for example differ by their RF frequency (e.g. RF1) and thus may target different layers of bodily tissues. For example:

A first work mode may be suitable for deep-tissue treatment and may correspond with a relatively low RF frequency: Experimental results have indicated optimal results for deep-tissue treatment for with a relatively low RF frequency (e.g. 0.8 MHZ).

A second work mode may be suitable for mid-layer tissue treatment. Experimental results have indicated optimal results for this type of treatment with an intermediate RF frequency (e.g. 1.7 MHZ).

A third work mode may be suitable for superficial tissue treatment. Experimental results have indicated optimal results for this type of treatment with a relatively high RF frequency (e.g. 2.45 MHZ).

A fourth work mode may, for example include a combination of the previous modes, to obtain a combined treatment of all aforementioned tissues. For example, RF generator 130 may be configured to emit an RF signal including a combination of a first signal of a first RF frequency, and a second signal of a second RF frequency, and a third signal of a third RF frequency.

Processor 120 may be configured to control RF generator 130, in accordance with users configurations, to apply these configurations during the RF treatment. Processor 120 may receive from a user (e.g. via UI 30) a set of required physical parameters (e.g. RF frequency RF1, current pulse amplitude I2, current pulse duration ΔT3 etc.), corresponding to a required RF current pulse P2.

Processor 120 may control RF generator 130 to produce a first RF current pulse P1, hereby referred to as a test RF current pulse, having a second set of parameters, hereby referred to as test parameters (e.g. RF frequency RF1, current pulse amplitude I1, current pulse duration ΔT1).

During emittance of test RF current pulse P1, Processor 120 may obtain at least one measurement of at least one electric parameter of the treated tissue (e.g. tissue impedance) from at least one sensor (e.g. electronic sensor 110 of FIG. 1), as elaborated below, in relation to FIG. 4.

Processor 120 may receive (e.g.: via UI 30, or hard-coded in an instruction code of processor 120) a predefined range for operation, in respect to one or more electric parameter of the treated tissue. In some embodiments, the predefined range may be a safety range defined empirically to avoid inflicting damage to the treated bodily tissue.

For example, a parameter of tissue resistance (R) may be required to be above a predefined threshold for the purpose of treatment, in order to avoid dissipating excessive electric power (P) thereto, due to the powers inverse relation with the resistance: P=V²/R (where V is the applied voltage).

If the value of the at least one electric parameter (e.g. tissue impedance) measured during the emittance of test RF current pulse P1 is within the predefined range, processor 120 may control RF generator 130 to produce at least one second RF current pulse P2, having a second set of physical parameters, according to the required set of physical parameters. Second RF current pulse P2 is hereby referred to as treatment RF current pulse P2.

For example, processor 120 may calculate the Root-Mean-Square voltage (Vrms) V2 required, according to the obtained measurement (e.g. tissue impedance) to produce a treatment RF current pulse P2, having the same amplitude as the RF current pulse required by the user. Processor 120 may configure RF generator 130 accordingly (e.g.: set RF generator's 130 output voltage), to emit the treatment RF current pulse required by the user.

Test pulse P1 may serve to affirm the condition of the treated tissue, prior to applying treatment pulse P2. Accordingly, test pulse P1 may dissipate a smaller amount of energy to the treated tissue, in comparison to treatment pulse P2. For example, test pulse P1 may be shorter in time (e.g. the duration of ΔT1 may be 10 milliseconds, whereas the duration of ΔT1 may be 200 milliseconds). In another example, the RMS voltage of treatment pulse P2 may be higher than that of test pulse P1.

If the value of the at least one electric parameter (e.g. tissue impedance) is beyond the predefined range (e.g. below a predefined threshold), processor 120 may control RF generator 130 to not produce the at least one treatment RF current pulse. For example, processor 120 may inactivate RF generator 130, or alternately nullify output voltage V2.

According to some embodiments, processor 120 may be configured to monitor, during the production of treatment RF current pulse P2, a value of at least one parameter of the treated tissue (e.g. impedance, resistance, temperature, etc.), as measured by the at least one sensor (e.g. elements 110 and 140). If the value of the at least one parameter is beyond a predefined range (e.g. impedance is beyond a predefined safety range, temperature is beyond a predefined threshold, etc.) processor 120 may control RF generator to halt the production of the at least one treatment RF current pulse P2. For example, processor 120 may: (a) store (e.g. in memory module 121) a value of a first measurement of the at least one electric parameter of the treated tissue during an initial period (e.g.: ΔT4) of pulse P2; (b) continuously and/or repetitively measure the at least one electric parameter throughout the duration of pulse P2; and (c) if a measurement of the at least one electric parameter differs from the stored value beyond a predefined threshold, then control RF generator to halt the production of the at least one treatment RF current pulse P2.

In some embodiments, processor 120 may control RF generator 130 to adjust at least one value of a physical parameter of the at least one treatment RF current pulse, according to (a) the monitored value of at least one electric parameter of the treated tissue, and (b) the required set of physical parameters. Pertaining to the example above: if processor 120 identifies a difference between a measurement of the at least one electric parameter (e.g. tissue impedance) and the stored value, that exceeds a predefined threshold, processor 120 may control RF generator 130 to adjust at least one parameter of the RF generator 130 configuration (e.g. output voltage) to comply with the parameters of the RF current pulse (e.g. current pulse amplitude), as required by the user.

Reference is now made to FIG. 4, which is a schematic block diagram depicting an embodiment of an RF generator 130, which may be included in a system for RF treatment according to some embodiments.

RF generator 130 may be communicatively connected to processor 120 and may receive at least one command therefrom. The command may include at least one parameter corresponding with a required functionality of RF generator 130. For example:

a first parameter may be a status of the RF generator, including one of active and non-active;

RF generator may be configured to be activated or deactivated accordingly;

and at least one second parameter may include at least one value of a physical parameter of an RF current pulse to be produced by the RF generator. The at least one second parameter may include at least one of: RF frequency, RF pulse duration, RF voltage amplitude and RF current amplitude.

RF generator 130 may be configured receive the command from processor 120 and produce at least one RF current signal according to the at least one parameter of the command.

RF generator 130 may include a Direct Digital Synthesis (DDS) module 134, configured to receive at least one parameter of the command, corresponding with a required RF frequency. DDS 134 may produce an RF waveform signal in the received frequency, as known to persons skilled in the art of signal processing. The RF waveform signal may be propagated to the VCG amplifier module 132.

RF generator 130 may include a Digital-to-Analog Converter (DAC) 131, and a Voltage Control Gain (VCG) amplifier 132. DAC 132 may receive at least one digital signal, corresponding to a value of a required output voltage amplitude. DAC 132 may convert the digital signal to an analog amplitude signal and may propagate the analog amplitude signal to the VCG amplifier 132.

VCG amplifier 132 may receive the analog amplitude signal and the waveform signal and produce an RF current signal, corresponding to parameters of RF frequency and amplitude, as included within the processor's command.

According to some embodiments, the RF current signal may be directly propagated to an RF emission portion (e.g. element 220 of FIG. 1), to be applied to a bodily tissue via a first and second electrode (e.g. elements 230, 240 of FIG. 1).

The term ‘derivative’ is used herein in reference to at least one signal that corresponds with the RF current signal but is not necessarily identical thereto.

In some embodiments, the RF current signal may be propagated from VCG 132 via at least one low-voltage isolation transformer T1 135 to produce a derivative of the RF current signal. The derivative may be propagated therefrom to RF emission portion 220, to be applied to a bodily tissue as described above.

In another embodiment, the derivative of the RF current signal may be propagated via at least one RF amplifier module 136 to RF emission portion 220. The at least one RF amplifier module 136 may reside within a back-end unit (e.g. element 100 of FIG. 1) or within a treatment device (e.g. element 200 of FIG. 1).

In yet another embodiment, the derivative of the RF current signal may be propagated via at least one high-voltage isolation transformer T2 137 to RF emission portion 220. The at least one transformer T2 137 may reside within a back-end unit (e.g. element 100 of FIG. 1) or within a treatment device (e.g. element 200 of FIG. 1).

The electric impedance of the treated tissue is schematically marked Z_load, and the combined output impedance of RF generator 130 and RF emission portion 220 is schematically marked Z_out, as known by convention to persons skilled in the art of electric engineering. In some embodiments, RF generator 130 may undergo a process of calibration, to determine the value of Z_out.

For example, RF generator may be calibrated according to the following steps:

Electrodes 230 and 240 may be connected via a known impedance (e.g. Z_calibration).

RF generator may be configured to produce at least one calibration RF current pulse, having at least one predefined set of parameters. The predefined set of parameters may include for example: RF frequency and voltage amplitude.

Electronic sensor 110 may be configured to measure an electric current conveyed through electrodes 230 and 240 (e.g. via known impedance Z_calibration) and propagate the measurement results to processor 120.

Processor 120 may calculate Z_out according to the predefined parameters of the at least one calibration RF current pulse (e.g. RF frequency and voltage amplitude) and the electric current measured by electronic sensor 110, according to Ohm's law, as known to persons skilled in the art of electric engineering.

Processor 120 may store (e.g. in memory module 121 of FIG. 1) the value of Z_out as determined during the process of calibration, for use during applying RF treatment to a bodily tissue.

In some embodiments, a sensor (e.g. electronic sensor 110 of FIG. 1) may include electronic sensor 110. When RF treatment system 10 is utilized to apply RF current pulses to a patient, electrodes 230 and 240 are not connected via a known impedance (e.g. Z_calibration). Processor 120 may therefore continuously and/or periodically monitor the current measured by electronic sensor 110, determine the overall impedance (e.g. Z_out+Z_load), and subtract the stored value of Z_out therefrom, to determine at least one momentary value of Z_load (e.g. an impedance of the treated bodily tissue).

As explained above in relation to FIG. 3, processor 120 may configure RF generator 130 to emit the at least one RF current pulse or halt the emittance thereof according to the measured impedance and/or current.

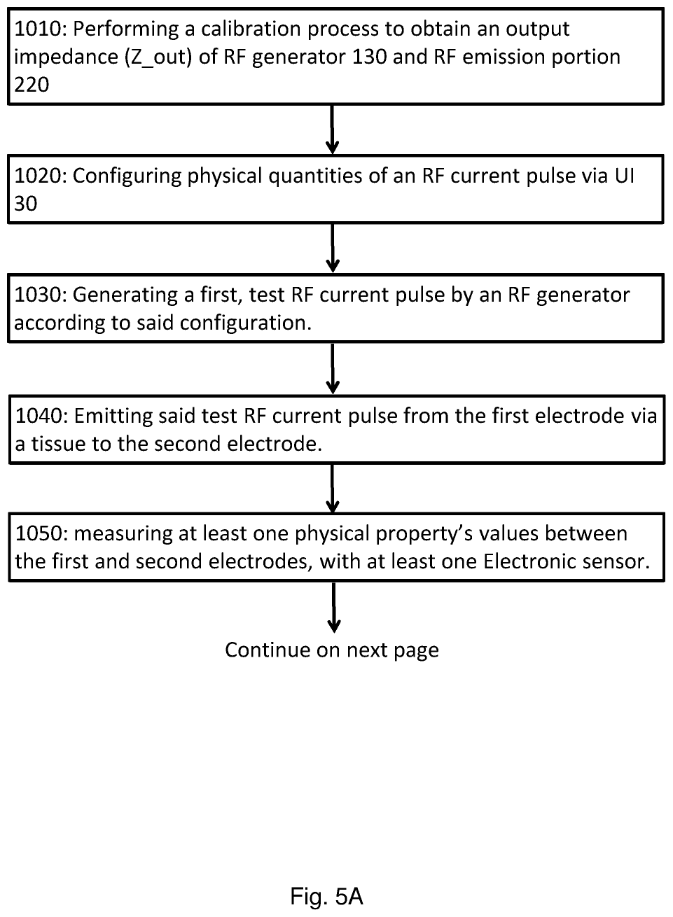

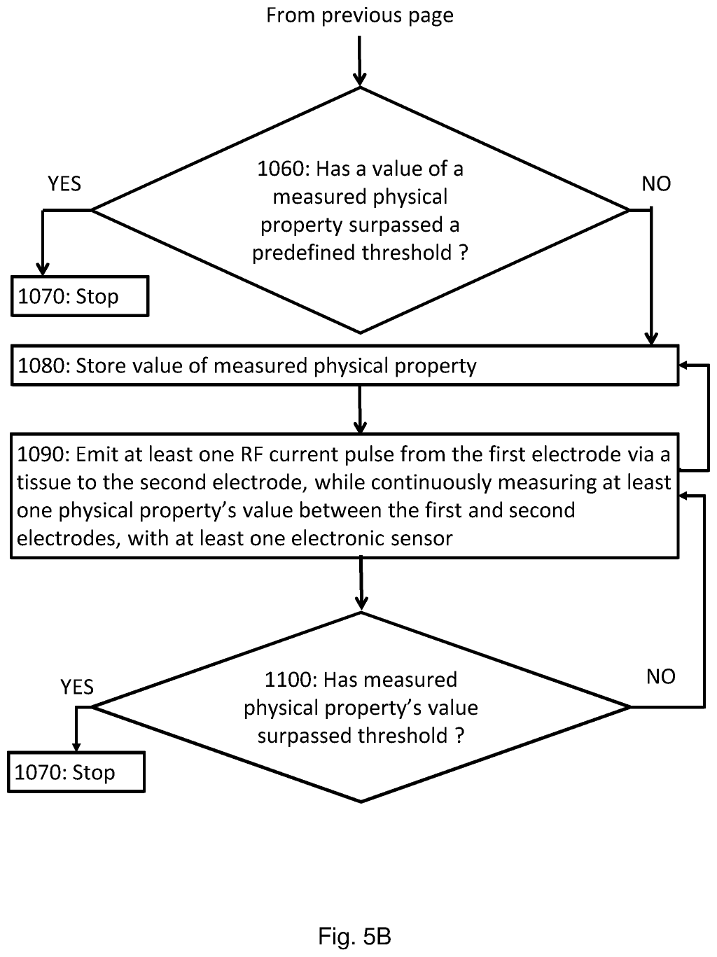

Reference is now made to FIGS. 5A and 5B that jointly show a flow diagram, depicting a method for utilizing RF treatment system (e.g. element 10 of FIG. 1) to apply RF treatment to a bodily tissue, according to some embodiments.

In step 1010, a calibration process may be performed (e.g. as explained above in relation to FIG. 4) to determine the output impedance (e.g. element Z_out of FIG. 4) of RF generator 130 and RF emission portion 220. The determined output impedance (e.g. Z_out) may be stored by processor 120 (e.g. on element 121 of FIG. 1).

In step 1020, UI module 30 may enable a user to configure at least one physical parameter of at least one RF current pulse, including for example: at least one voltage amplitude, at least one current amplitude, an RF frequency, an RF pulse width, a pulse frequency, a pulse duty-cycle, a number of pulses in a pulse-train, an overall energy dissipated by the electrodes, and a duration of an RF treatment.

In step 1030, processor 120 may control RF generator 130 to generate a single, test RF current pulse according to at least one configured physical parameter. The test RF current pulse may be configured to enable the system to ascertain whether the treatment may be commenced safely under said treatment configuration, or whether it should be halted, to prevent damage to the treated tissue.

In some embodiments, the amplitude of the test RF current pulse may be set lower than the amplitude of the configured physical parameter by a predefined percentage (e.g. 50%). In some embodiments, the duration of the test RF current pulse may be set shorter than the duration of the configured physical parameter. For example, the duration of the test RF current pulse may be less than 10 milliseconds, whereas a typical RF current pulse for RF treatment may span more than 100 milliseconds.

The amplitude and duration of the test RF current pulse may be set according to empirical values so as to avoid any damage to the treated tissue. According to experimental results, an RMS voltage amplitude of 40 volts and a duration in the range of 6-12 milliseconds may accommodate this requirement.

In step 1040, the test RF current pulse may be emitted from the first electrode 230 via the treated tissue to the second electrode 240. The current density of the RF current pulse through the tissue may correspond to the ratio between the contact surfaces of the first and second electrodes with the tissue. For example, the current density at the location of first electrode's 230 contact surface may be higher than the current density at the location of second electrode's 240 contact surface. Consequently, the treated tissue is heated at the location of the first electrode more intensely than at the location of the second electrode.

In step 1050, At least one sensor (e.g. electronic sensor 110 of FIG. 1, element 133 of FIG. 4) may continuously and/or repetitively measure values of physical properties (e.g. current amplitude, electric resistance, electric impedance) of the treated tissue, between the first and second electrodes, as explained above in relation to FIG. 4.

In some embodiments, as explained above in relation to FIG. 4, electronic sensor 110 may be configured to measure a current amplitude, and processor 120 may be configured to calculate at least one physical property (e.g. electric impedance) of the treated tissue according to the measured current, configured voltage amplitude, configured RF frequency and determined output impedance (e.g. Z_out).

In step 1060, if a measured physical property value (e.g. electric impedance) surpasses a predefined threshold (e.g. if the electric impedance of the treated tissue is beyond a predefined range), then processor 120 may command RF generator 130 to stop the treatment, and no further RF current pulses may be dissipated into the treated tissue (step 1070). For example, a high value of calculated load resistance may indicate a condition in which one of the electrodes is in poor contact with the tissue. This condition may result in damage to the treated tissue and requires abrupt termination of the treatment. In another example, if a temperature sensed by a temperature sensor (e.g. element 140 of FIG. 1) surpasses a predefined threshold, the patient may be experiencing over-heating caused by excessive power dissipation into the treated tissue. This condition may also require abrupt termination of the treatment.

In step 1080, values of physical properties (e.g. calculated load resistance) measured during the test pulse may be stored in a computer memory (e.g. element 121 of FIG. 1), associated with the processor.

In step 1090, if the value of the at least one measured physical property has not surpassed the predefined thresholds, the process may be allowed to proceed. Processor 120 may command RF generator 130 to emit at least one RF current pulse from the first electrode via the treated tissue to the second electrode. The at least one electronic sensor 110 may continuously and/or repeatedly measure a value of at least one physical property of the treated tissue (e.g. RMS load voltage) between the first and second electrodes (e.g.: 230 and 240 respectively).

The duration of the at least one treatment pulse may be longer than the duration of the test pulse. According to some embodiments, the duration of the treatment pulse may be in the range of 100 milliseconds to 300 milliseconds.

In step 1100, processor 120 may continuously and/or repeatedly monitor the measurements performed by the at least one electronic sensor 110, to determine whether a value of at least one monitored physical property has changed beyond a predefined threshold and control the function of the RF generator 130. For example, if the calculated load resistance has surpassed a predefined threshold or has incremented by a value that surpasses a predefined threshold—the treatment may be stopped.

According to some embodiments, the processor may be configured to monitor the measurements of physical properties by comparing a plurality of measurements throughout the duration of the treatment RF pulse. According to these embodiments, the treatment RF pulse may be divided to a plurality of segments, and measurements may be performed repetitively, in relation to each of these segments. For example: a single treatment RF pulse may be 200 milliseconds long, and each segment may be 2 milliseconds long, hence the measurements may be performed 100 times throughout the duration of the treatment RF pulse. Measurement of physical properties relating to the first segment (e.g. first 2 ms) of at least one treatment RF pulse may be stored in a computer memory (e.g. element 121 of FIG. 1). Processor 120 may compare subsequent measurements, relating to subsequent segments of the treatment RF pulse, to the measurement of the first segment, and detect a condition in which the values of at least one physical property (e.g. impedance of a treated tissue) has changed beyond a predefined threshold throughout the duration of the treatment RF pulse.

According to some embodiments, if a value of a measured physical property has surpassed a predefined threshold, beyond a predefined tolerance range, processor 120 may control the RF generator to stop emitting treatment RF current pulses and may terminate the treatment. However, if the value of a measured physical property has surpassed a predefined threshold but is within a predefined tolerance range, processor 120 may adapt the configuration of RF generator 130 and may continue to emit the at least one treatment RF current pulse. For example, throughout the treatment process, the processor may control the RF pulse voltage amplitude, so as to keep the current within a predefined safe range according to the calculated load resistance.

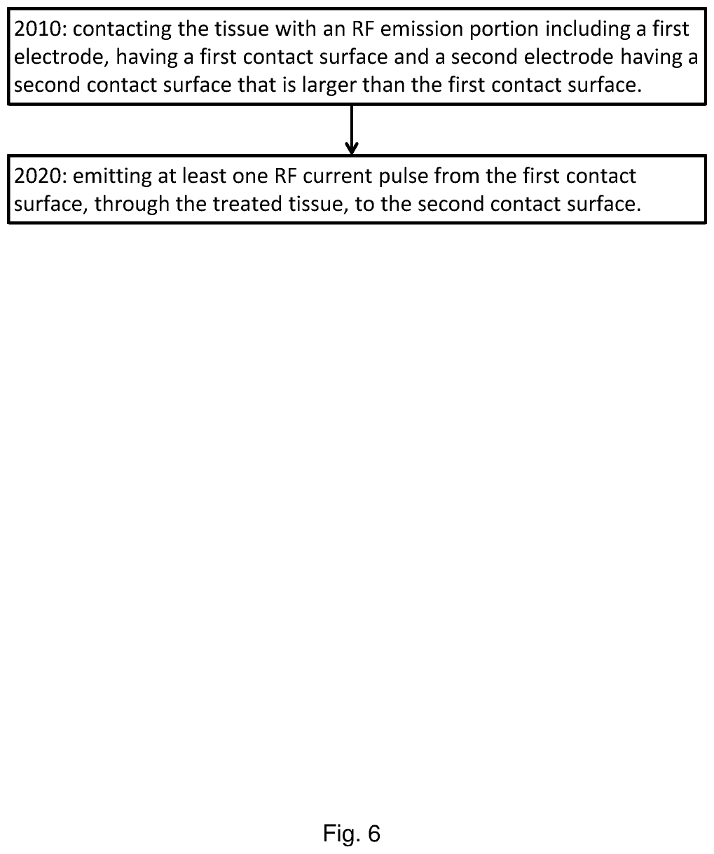

FIG. 6 shows a flow diagram, depicting a method for employing RF treatment, according to some embodiments.

In step 2010, embodiments may include contacting a treated tissue with an RF emission portion 220. RF emission portion 220 may include a first electrode, having a first contact surface and a second electrode having a second contact surface that is larger than the first contact surface.

In step 2020 embodiments may include emitting at least one RF current pulse from the first contact surface, through the treated tissue, to the second contact surface.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A device for radio frequency (RF) treatment of a tissue, the device comprising: a hand-held portion and an RF emission portion coupled to the hand-held portion, wherein the RF emission portion comprises: a first electrode, having a first contact surface and a second electrode having a second contact surface that is larger than the first contact surface, and wherein the first electrode is configured to emit at least one RF current pulse from the first contact surface, through the tissue to be treated, to the second contact surface.

2. The device of claim 1, further comprising an insulating medium, placed between the first electrode and the second electrode, and wherein the second electrode serves as a reference voltage node to the first electrode, and wherein the contact surface of the second electrode is larger than the contact surface of the first electrode by at least a factor of 3.

3. The device of claim 1, further comprising at least one first sensor, configured to measure the value of at least one electric parameter of the treated tissue, wherein said parameter is at least one of: voltage, current, power, energy, resistance and impedance.

4. The device of claim 3, further comprising a second sensor, configured to measure the temperature of the treated tissue.

5. The device of claim 1, wherein the hand-held portion comprises:
a hollow portion having an opening toward the treated tissue, and configured to accommodate a liquid; and
an actuator associated with the hollow portion,
wherein the actuator is configured to eject at least part of the liquid out of the hollow portion, through the opening onto the treated tissue.

6. The device of claim 2, wherein a first RF emission portion is removably coupled to the hand-held portion and is interchangeable with a second RF emission portion, and wherein the first and second RF emission portions differ by at least one physical property, and wherein said property is at least one of: a ratio between the area of contact surfaces of the first and second electrodes, a size of at least one of first and second electrodes and a shape of at least one of first and second electrodes.

7. The device of claim 1, configured to apply RF energy to a vaginal tissue.

8. A system for RF treatment of a tissue, the system comprising a hand-held portion and an RF emission portion coupled to the hand-held portion, wherein the RF emission portion comprises: a first electrode, having a first contact surface and a second electrode having a second contact surface that is larger than the first contact surface, and wherein the first electrode is configured to emit at least one RF current pulse from the first contact surface, through the treated tissue, to the second contact surface.

9. The system of claim 8, further comprising an insulating medium, placed between the first electrode and the second electrode, and wherein the second electrode serves as a reference voltage node to the first electrode, and wherein the contact surface of the second electrode is larger than the contact surface of the first electrode by at least a factor of 3.

10. The system of claim 8, further comprising at least one RF generator, configured to produce at least one RF electric current pulse, and provide the at least one pulse to the RF emission portion, and wherein the RF emission portion is configured to apply the at least one pulse to the treated tissue via the electrodes.

11. The system of claim 10 further comprising a processor, communicatively connected to the RF generator, wherein the processor is configured to control a status of the RF generator, wherein said status is one of active and non-active, and wherein the processor is configured to control at least one value of a physical parameter of the at least one RF current pulse produced by the RF generator.

12. The system of claim 11 wherein the at least one physical parameter is at least one of: RF frequency, RF pulse duration, and RF voltage amplitude.

13. The system of claim 12 further comprising at least one of:
a first sensor, configured to measure the value of at least one electric parameter of the treated tissue, wherein said parameter is at least one of a list consisting: voltage, current, power, energy, resistance and impedance; and
a second sensor, configured to measure the temperature of the treated tissue.

14. The system of claim 13, wherein the processor is communicatively connected to at least one sensor and is configured to obtain measured data therefrom, and wherein the processor is further configured to control at least one of the status of the RF generator and the value of the at least one physical parameter according to the obtained measured data.

15. The system of claim 14, wherein the processor is configured to:
receive from a user, via a user interface, a required set of physical parameters, corresponding to a required RF current pulse;
control the RF generator to produce a first RF current pulse, having a test set physical parameters;
obtain at least one measurement of at least one electric parameter of the treated tissue from at least one sensor;
if the value of the at least one electric parameter is within a predefined range, then control the RF generator to produce at least one second RF current pulse, having a second set of physical parameters, according to the required set of physical parameters; and
if the value of the at least one electric parameter is beyond the predefined range, control the RF generator to not produce the at least one second RF current pulse.

16. The system of claim 15, wherein the processor is further configured to:
monitor, during the production of the at least one second RF current pulse, a value of at least one electric parameter of the treated tissue, as measured by the at least one sensor; and
if the value of the at least one electric parameter is beyond the predefined range, control the RF generator to halt the production of the at least one second RF current pulse.

17. The system of claim 16, wherein the processor is further configured to control the RF generator to adjust at least one value of a physical parameter of the at least one second RF current pulse, according to (a) the monitored value of at least one electric parameter of the treated tissue, and (b) the required set of physical parameters.

18. The system of claim 14, wherein the hand-held portion comprises:
a hollow portion having an opening toward the tissue to be treated, and configured to accommodate a liquid; and
an actuator, associated with the hollow portion, and electrically connected to the processor,
wherein the actuator is configured to, upon receiving a command from the processor, eject at least part of the liquid out of the hollow portion, through the opening onto the tissue to be treated.

19. A method of applying RF treatment to a tissue, the method comprising:
contacting the tissue with an RF emission portion comprising: a first electrode, having a first contact surface and a second electrode having a second contact surface that is larger than the first contact surface and;
emitting at least one RF current pulse from the first contact surface, through the treated tissue, to the second contact surface.

20. The method of claim 19, further comprising placing an insulating medium between the first electrode and the second electrode and having the second electrode serve as a reference voltage node to the first electrode, and wherein the contact surface of the second electrode is larger than the contact surface of the first electrode by at least a factor of 3.
